(19) United States
(12) Patent Application Publication
(10) Pub. No.: US 2017/0109563 A1
(43) Pub. Date: Apr. 20, 2017

(54) PALM VEIN-BASED LOW-COST MOBILE IDENTIFICATION SYSTEM FOR A WIDE AGE RANGE

(71) Applicant: WAYNE STATE UNIVERSITY, Detroit, MI (US)

(72) Inventors: Paul E. KILGORE, Bloomfield Hills, MI (US); Weisong SHI, Troy, MI (US); Jie CAO, Royal Oak, MI (US); Zhifeng YU, Oakland, MI (US); Mingyang XU, Raleigh, NC (US)

(21) Appl. No.: 15/293,798

(22) Filed: Oct. 14, 2016

Related U.S. Application Data
(60) Provisional application No. 62/241,500, filed on Oct. 14, 2015.

(51) Int. Cl. G06K 9/00; G06F 19/00; G06T 7/00; G06K 9/46; H04N 5/33; G06K 9/20

(52) U.S. Cl. CPC G06K 9/00087; H04N 5/33; G06K 9/2027; G06T 7/0081; G06K 9/46; G06T 7/0044; G06F 19/322; G06K 2009/4666; G06T 2207/20112

(57) ABSTRACT

A system includes an infrared camera, an infrared light source, and a processor. The processor is programmed to receive, from the infrared camera, an image of a hand illuminated using the infrared light source, and send the image to a remote server to identify a user corresponding to the hand according to a vein pattern of the hand. An image of a hand illuminated using an infrared light source is received from an infrared camera. Region-of-interest segmentation is performed on the image to generate a segmented image of consistent hand location and orientation. Feature extraction is performed on the segmented image to generate a feature-extracted vein image. Matching of the feature-extracted vein image is performed against a database of feature-extracted vein images to identify a user identity corresponding to the hand.

### CROSS-REFERENCE TO RELATED APPLICATIONS

This application claims the benefit of U.S. provisional application Ser. No. 62/241,500 filed Oct. 14, 2015, the disclosure of which is hereby incorporated in its entirety by reference herein.

### TECHNICAL FIELD

Aspects of the disclosure generally relate to mobile identification of individuals across a wide age range according to palm veins.

### BACKGROUND

Traditional methods of access control, such as token-based identification methods (e.g., an ID card or passport) and knowledge-based identification methods (e.g., a password), are being replaced by biometrics recognition technology in many fields. This change is occurring due to limitations in reliability and usability of passwords and cards. In some situations, biometric-based authentication is more reliable compared to traditional methods to control access.

### SUMMARY

In one or more illustrative embodiments, a system includes an infrared camera, an infrared light source, and a processor. The processor is programmed to receive, from the infrared camera, an image of a hand illuminated using the infrared light source, and send the image to a remote server to identify a user corresponding to the hand according to a vein pattern of the hand.

In one or more illustrative embodiments, an image of a hand illuminated using an infrared light source is received from an infrared camera. Region-of-interest segmentation is performed on the image to generate a segmented image of consistent hand location and orientation. Feature extraction is performed on the segmented image to generate a feature-extracted vein image. Matching of the feature-extracted vein image is performed against a database of feature-extracted vein images to identify a user identity corresponding to the hand.

In one or more illustrative embodiments, a system includes a computing device, including a processor and a memory. The computing device is programmed to execute instructions stored to the memory to receive, from an infrared camera, an image of a hand illuminated using an infrared light source; perform region-of-interest segmentation on the image to generate a segmented image of consistent hand location and orientation; perform feature extraction of the segmented image to generate a feature-extracted vein image; and match the feature-extracted vein image against a database of feature-extracted vein images to identify a user corresponding to the hand.

### BRIEF DESCRIPTION OF THE DRAWINGS

FIG. 1 illustrates an example system for low-cost, portable, and child-friendly identification of individuals implemented using vein pattern recognition;

FIG. 2 illustrates an example detail of a mobile device including an embedded camera;

FIG. 3 illustrates an example detail of a camera including an infrared filter;

FIG. 4 illustrates an example process for vein pattern recognition;

FIG. 5 illustrates an example process for feature extraction performed for vein pattern recognition;

FIG. 6 illustrates an example image of a hand captured by the camera and sent to the remote server from the mobile device;

FIG. 7 illustrates an example of an illuminate vein system using light transmission;

FIG. 8 illustrates an example of a illuminate vein system using light reflection;

FIG. 9 illustrates an example of an image capture of a hand by the mobile device including the camera using the light source;

FIG. 10 illustrates an example infrared flashlight light source;

FIG. 11 illustrates a diagram including an example region of interest overlaid on a representation of a hand;

FIG. 12 illustrates an example diagram of stages of feature extraction;

FIG. 13 illustrates an example diagram of registration of vein pattern;

FIG. 14 illustrates an example diagram of thinning of feature-extracted images; and

FIG. 15 illustrates an example diagram of patterns of a non-single pixel point.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

In the health care arena, there are a number of applications that require biometric recognition of their subjects. For example, in some developing countries where there is no valid civic ID system, immunization records may be tracked using biometrics. The biometric recognition may be performed for users of different ages, as well as for users that age over time.

Fingerprint biometrics are widely used from civil records management to smart phone authentication. However, performance of fingerprint is unsatisfying when skin condition of the finger is not good (dry, dirty, or scarred). Moreover, fingerprints can be easily copied from touch trace or even photo. Retinal scanning and iris recognition are very accurate and difficult for replication, but a device for performing the eyeball scan is relatively expensive. Other biometrics technologies such as facial recognition and voice recognition are affordable, but their accuracy is not adequate for precise applications, for instance, health records, or gateway access control.

As explained in detail herein, vein pattern identification may be utilized in patient identification. By using vein pattern identification, a health care system may reduce redundant health records, prevent medical errors, reduce fraud at the point of service, and facilitate delivery of efficient and precise medical service.

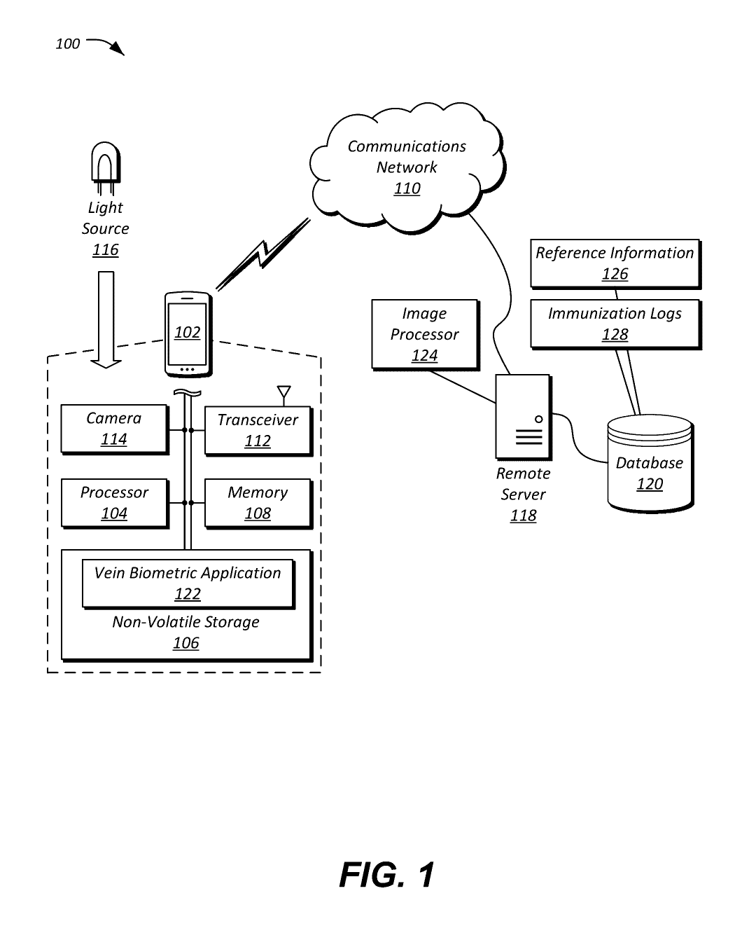

FIG. 1 illustrates an example system 100 for low-cost, portable, and child-friendly identification of individuals implemented using vein pattern recognition. The system includes a mobile device 102 in communication with a remote server 118 over a communication network 110. The mobile device 102 includes a camera 114 to capture an image of a hand of an individual. The system further includes a light source 116 for illuminating the hand. The mobile device 102 also includes a processor 104 and storage 106 onto which a vein biometric application 122 is installed. The vein biometric application 122 is programmed to cause the camera 114 to capture and send the image to the remote server 118 over the network 110. The remote server 118 includes an image processor 124 configured to receive and perform feature extraction of the captured image to generate a feature-extracted vein image, and access a database 120 of feature-extracted vein images (i.e., reference information 126) against which the captured and feature-extracted vein image may be quickly matched for identification of the individual. Identification of the individual may accordingly allow for immunization logs 128 or other records relating to the identified individual to be identified. While an example system 100 is shown in FIG. 1, the example components as illustrated are not intended to be limiting. Indeed, the system 100 may have more or fewer components, and additional or alternative components and/or implementations may be used. As some examples, some or all of the operations performed by the remote server 118 and/or database 120 may be performed by the mobile device 102, and/or by a laptop or other computing device local to the mobile device 102.

The mobile device 102 may be of various types of portable computing device, such as cellular phones, tablet computers, smart watches, laptop computers, portable music players, or other devices. The mobile device 102 may further include various types of computing apparatus in support of performance of the functions of the mobile device 102 described herein. In an example, the mobile device 102 may include one or more processors 104 configured to execute computer instructions, and a storage medium 106 on which the computer-executable instructions and/or data may be maintained. A computer-readable storage medium 106 (also referred to as a processor-readable medium or storage 106) includes any non-transitory (e.g., tangible) medium that participates in providing data (e.g., instructions) that may be read by a computer (e.g., by the processor(s)). In general, a processor 104 receives instructions and/or data, e.g., from the storage 106, etc., to a memory 108 and executes the instructions using the data, thereby performing one or more processes, including one or more of the processes described herein. Computer-executable instructions may be compiled or interpreted from computer programs created using a variety of programming languages and/or technologies, including, without limitation, and either alone or in combination, Java, C, C++, C#, Fortran, Pascal, Visual Basic, Python, Java Script, Perl, PL/SQL, etc.

The communications network 110 may include one or more interconnected communication networks such as the Internet, a cable television distribution network, a satellite link network, a local area network, a wide area network, and a telephone network, as some non-limiting examples. Using a transceiver 112, the mobile device 102 may be able to send outgoing data from the mobile device 102 to network destinations on the communications network 110, and receive incoming data to the mobile device 102 from network destinations on the communications network 110. The transceiver 112 may include a cellular modem or other network transceiver configured to facilitate communication over the communications network 110 between the mobile device 102 and other devices of the system 100.

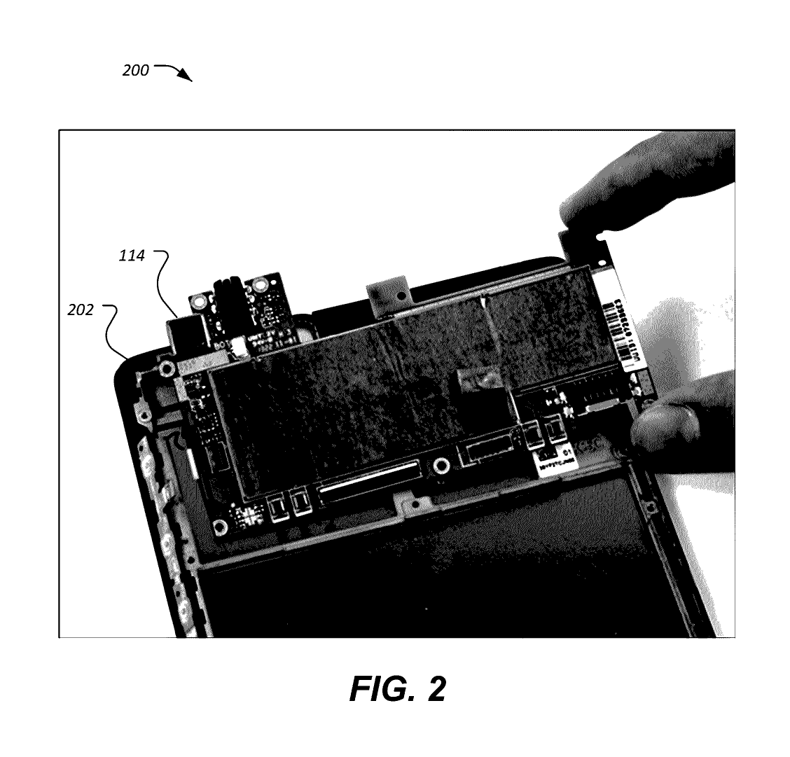

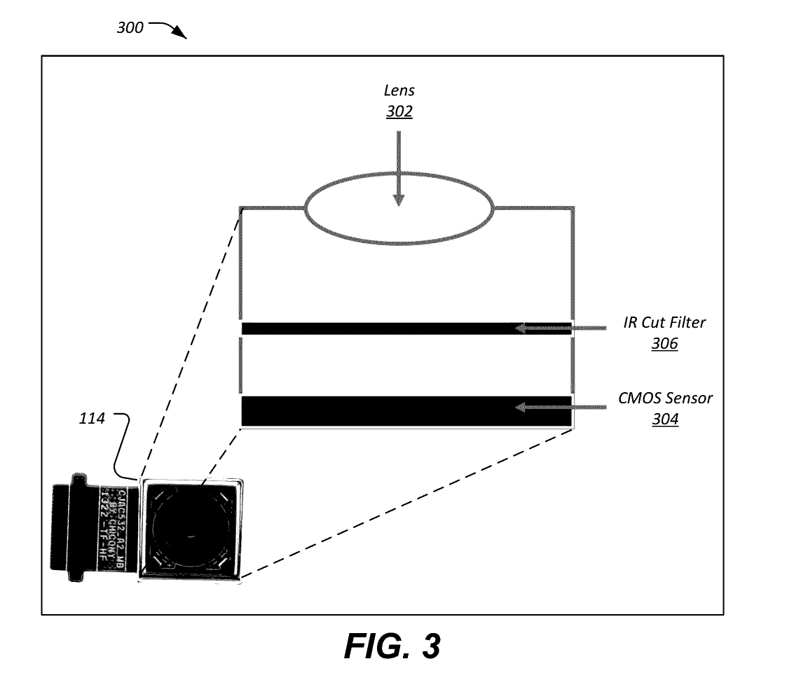

The mobile device 102 may include a camera 114 configured to capture images such as still photographs, and/or sequences of images such as video. Referring to FIG. 2, in one example, the mobile device 102 may be a Google Nexus 7 2^{nd} Generation Android tablet, although other examples are contemplated. Referring to FIG. 3, the camera 114 may include a lens 302 passing light to an embedded complementary metal-oxide-semiconductor (CMOS) sensor 304. In some examples, to improve the image quality, an infrared filter 306 (e.g., an 850 nanometer (nm) filter) may be installed to the camera 114. The filter 306 may be placed in the light path between the lens 302 and CMOS sensor 304, to configure the camera 114 to allow infrared light to go through and reject light of other frequencies. Accordingly, the filter 206 may aid the camera 114 element in eliminating interference from other sources, such as from natural light.

Referring back to FIG. 1, the light source 116 may be configured to provide light in support of the image capture functionality of the camera 114. In an example, the light source 116 may be a battery-powered flashlight, e.g., an infrared flashlight. As one possibility, the light source 116 may be a YeShiNeng 100B infrared flashlight, configured to provide 850 nm wavelength infrared light with five watt power. The light source 116 may further be configured to support multiple intensity settings, e.g., strong, medium, and weak. Different intensity levels may accordingly be applied for subjects with different thickness of hand palm.

The remote server 118 may include various types of computing apparatus, such as a computer workstation, a server, a desktop computer, a virtual server instance executed by a mainframe server, or some other computing system and/or device. As mentioned above, computing devices, such as the remote server 118, generally include a memory on which computer-executable instructions may be maintained, where the instructions may be executable by one or more processors of the computing device.

In some examples, the remote server 118 may include or be in communication with a database 120. Databases 120, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners. A file system may be accessible from a computer operating system, and may include files stored in various formats. An RDBMS generally employs the Structured Query Language (SQL) in addition to a language for creating, storing, editing, and executing stored procedures, such as the PL/SQL language mentioned above. The database 120 may be configured to store information, such as the reference information 126 and/or immunization logs 128.

The vein biometric application 122 may be one application included on the storage of the mobile device 102. The vein biometric application 122 may include instructions that, when executed by the processor 104 of the mobile device 102, cause the camera 114 of the mobile device 102 to capture an image, and transmit the image over the network 110 to the remote server 118 for processing.

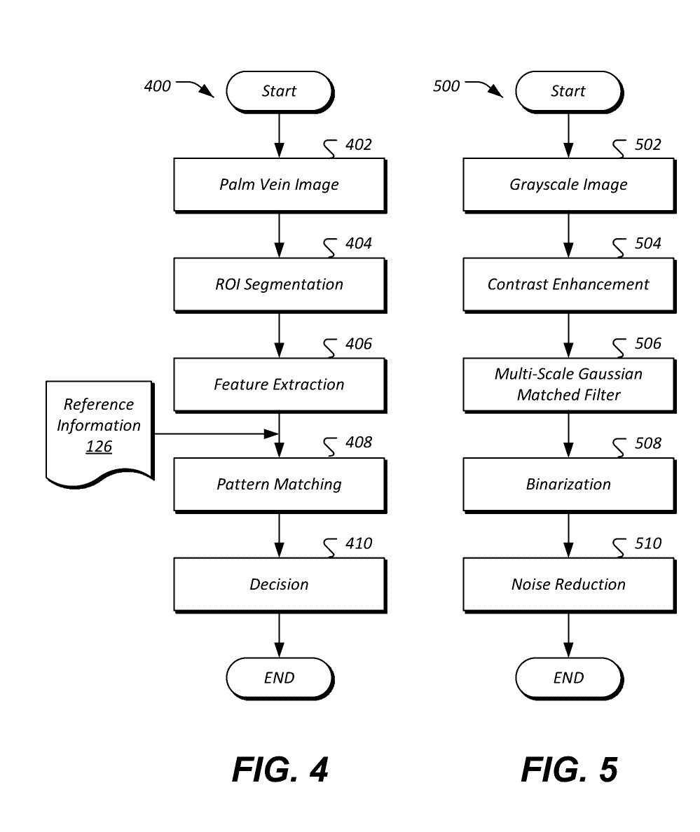

The image processor 124 of the remote server 118 may be configured to receive the image from the mobile device 102, access the reference information 126 to match the image to an identity, and retrieve immunization logs 128 or other information related to the identified user based on the matching. The image processor 124 may be implemented in a combination of hardware, software, and/or firmware executing on one or more processors of the remote server 118. Further aspects of the operation of the image processor 124 are discussed in detail below with respect to FIGS. 4-5.

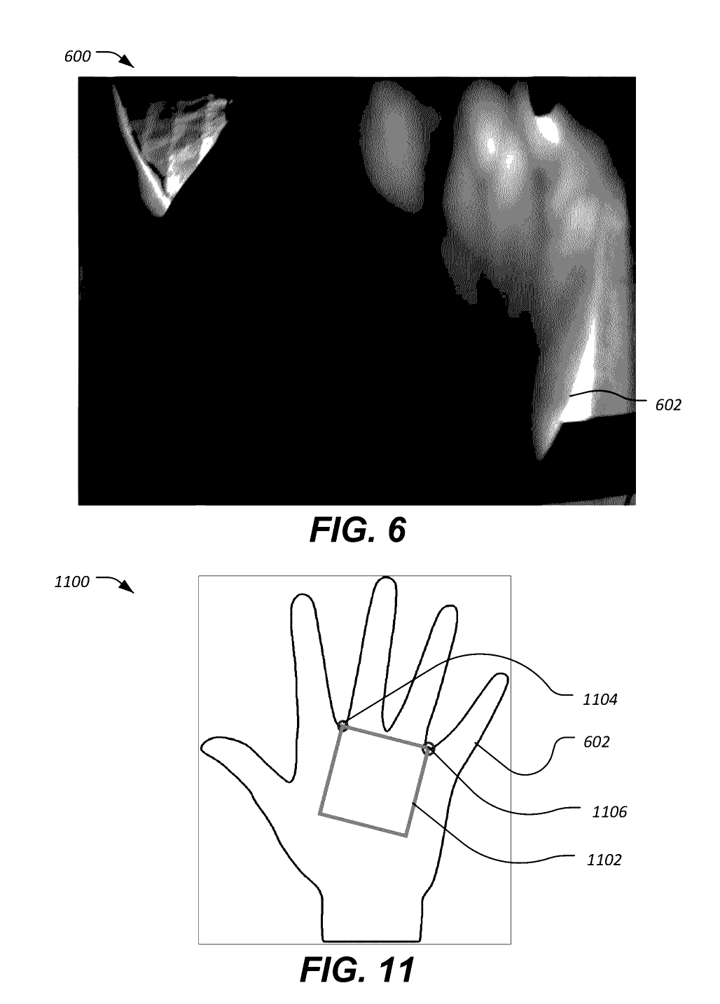

FIG. 4 illustrates an example process 400 for vein pattern recognition. In an example, the process 400 may be performed by the image processor 124 of the remote server 118. The process 400 may be initiated at 402, in an example, responsive to receipt from the mobile device 102 of an image for identification. FIG. 6 illustrates an example of one such image 600 of a hand 602 captured by the camera 114 and sent to the remote server 118 from the mobile device 102.

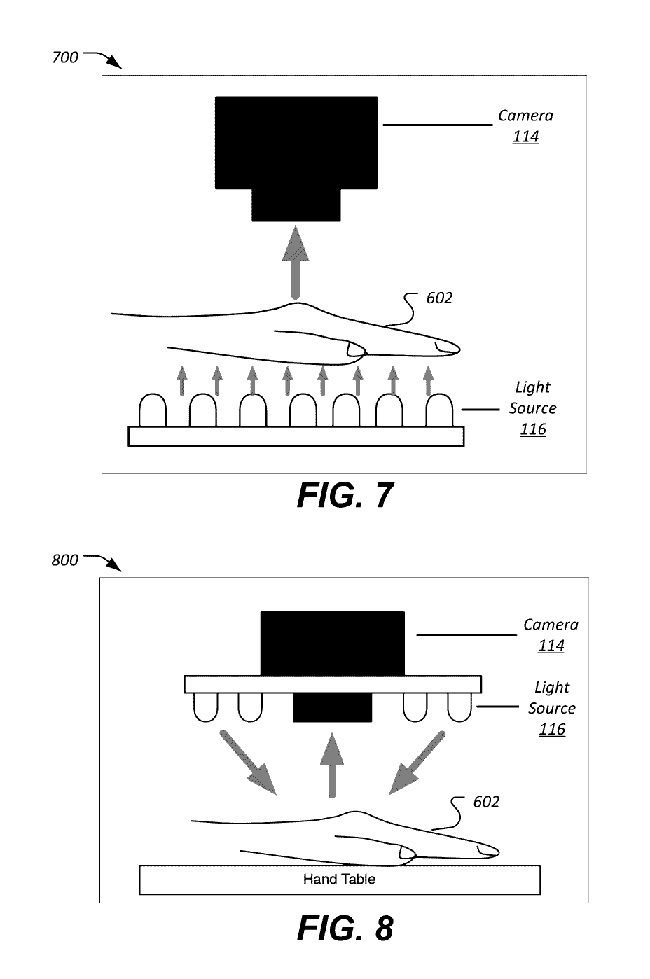

In some examples, such as those illustrated in FIGS. 7 and 8, a fixed relative position of the camera 114 to the hand being scanned may reduce differences in position. FIG. 7 illustrates an example 700 of an illuminate vein system using light transmission. As shown, the light source 116 is below the hand 602, and the camera 114 captures the image 600 of the hand 602 using the light transmitted through the hand 602 tissue. In contrast, FIG. 8 illustrates an example 800 of a illuminate vein system using light reflection. As compared to the example 700, in the example 800, the light source 116 and camera 114 are both above the hand 602, and the camera captures the image 600 of the hand 602 using light reflected from the surface of the hand 602 tissue.

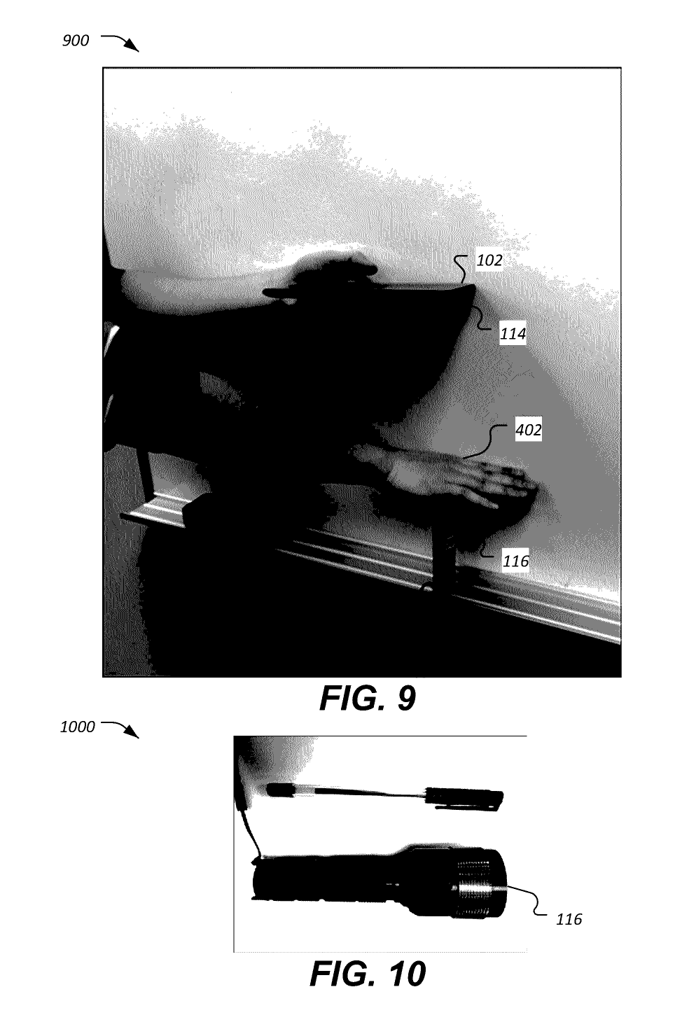

In still other examples, the light source 116, hand 602, and camera 114 may all be free to move with respect to one another. In such situations, the position of the hand could change in every captured image. FIG. 9 illustrates an example 900 of an image 600 capture of a hand 602 by the mobile device 102 including the camera 114 using the light source 116. In an example, the mobile device 102 may include the integrated infrared camera 114, and the light source 116 may be an infrared flashlight. An example infrared flashlight light source 116 is illustrated in FIG. 10 for size comparison with a pen 802. In a case of a child being imaged, the child may optionally grasp the flashlight or other light source 116 with his or her hand 602.

At operation 404 of FIG. 2, the image processor 124 performs region of interest (ROI) segmentation. For vein pattern recognition technology, it is important that the regions used for feature extraction for multiple visits from the same person come from a consistent place on the hand. Otherwise, error may be introduced into the matching process due to rotation or shifting of the vein pattern features between scans.

FIG. 11 illustrates a diagram 1100 including an example ROI 702 overlaid on a representation of a hand 602. The ROI 702 area shows an example of ROI segmentation for use in identifying vein pattern features. In the illustrated example, the image processor 124 detects the ROI 702 as the joint 704 between index finger and middle finger as well as the joint 706 between middle finger and ring finger from the infrared image, and draws a square based on the position of these two joint points 704, 706. This square space from the hand-dorsal may be referred to as the ROI 702 and may be used by the image processor 124 for the feature extraction. This ROI 702 segmentation may be robust to hand rotation and shift.

At operation 406 of FIG. 4, the image processor 124 performs feature extraction. Responsive to segmentation of the raw infrared hand 602 image, the image processor 124 performed vein system feature extraction upon the segmented image. Further aspects of the feature extraction are discussed below with respect to the process 500 of FIG. 5.

At 502, the image processor 124 converts the image into a grayscale image. In an example, the image, as ROI segmented, is normalized in size by the image processor 124. In an example, the image processor 124 normalizes the image from its initial dimensions (and potentially orientation) to a size of 256 pixels by 256 pixels. This normalization can save storage space in the database 120, accelerate image processing speed, and reduce deviation introduced by different size in the matching stage.

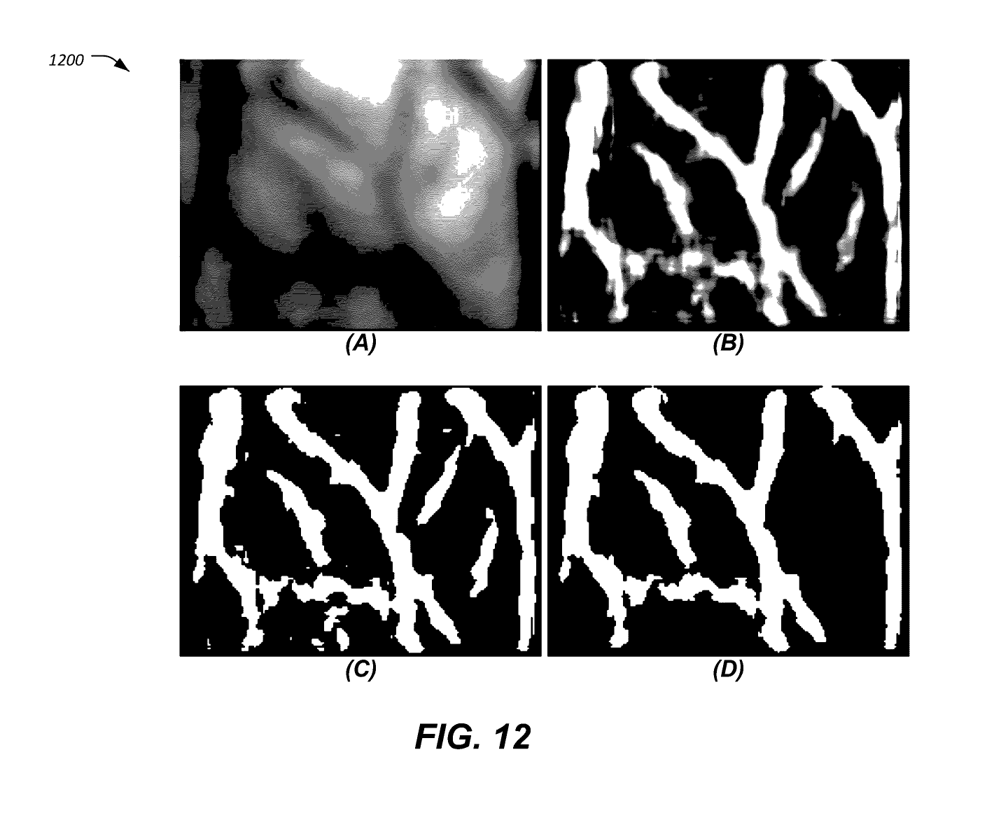

At 504, the image processor 124 applies a contrast stretching algorithm to enhance the contrast of the image. This may be performed, for example, to further clarify the vein patterns in the grayscale image. For instance, the image processor 124 may perform histogram equalization to change the original data from color image into a grayscale one. The histogram equalization may change the original color image to a grayscale for further processing and enhance the image contrast to make it easier for feature extraction. An example image converted to grayscale is shown as element (A) of the diagram 1200 of FIG. 12. It should be noted that in some examples, the operations of steps 502 and 504 may be performed utilizing the histogram equalization.

At 506, the image processor 124 employs a multi-scale Gaussian matched filter to extract the vein pattern lines from the background. The multi-scale Gaussian matched filter may be employed to extract the lines, as the cross sections of the image are similar to Gaussian shape lines. An example Gaussian matched filter may is defined in Equation 1, where Ø is the filter direction, and the values of Ø=0, Ø=pi/6, Ø=pi/4, Ø=pi/3, Ø=pi/2, Ø=3*pi/4, Ø=5*pi/6 are used to generate the filter in different directions and utilize median filters to reduce the noise, m is the mean value of the filter, a is standard deviation of filter, and L is the length of the filter in y direction.

$$\{ \begin{matrix}{ℊ}_{∅} ( x , y ) = - exp ( - \frac{{x}^{′2}}{{σ}_{x}^{2}} ) - m \\ {x}^{′} = x cos ∅ + y sin ∅ \\ {y}^{′} = y cos ∅ = x sin ∅\end{matrix} \qquad ( 1 )$$

The size of the multi-scale Gaussian matched filter can be adjusted by the constraint condition: |x′|=≦3sσₓ, |y′|≦sL/2. Production of the filter responses at different scales may be utilized to reduce noise. Element (B) of FIG. 12 illustrates an example response of the multi-scale Gaussian matched filter extraction of the vein pattern lines shown at element (A) of FIG. 12.

At 508, the image processor 124 performs binarization, in which the image after the multi-scale Gaussian matched filter is transferred from grayscale into a pure black and white. Element (C) of FIG. 12 illustrates an example binary image created from the vein pattern lines shown at element (B) of FIG. 12.

At 510, the image processor 124 employs a de-noise algorithm for noise reduction of the binary image. This may be because a binarized image containing clear vein information can be obtained using the filter responses, but may also include remaining noise in the image as shown at Element (C) of FIG. 12. To remove the remaining noise (e.g., noise elements having a small area), the image processor 124 may (i) search for unlabeled pixels, (ii) use a flood-fill algorithm to label all the pixels in the connected component, (iii) repeat operations (i) and (ii) until all the pixels are labelled, (iv) compute the area of each block of connected pixels, and (v) reduce the connected pixel areas which are below a threshold size. Element (D) of FIG. 12 illustrates an example de-noised image created from the binary image shown at element (C) of FIG. 12. After operation 510, control returns to operation 408 of the process 400.

Referring to FIG. 4, at 408 the image processor 124 performs pattern matching. The matching may be performed, in an example, using a captured image compared against reference information 126 of a plurality of known images of users, to identify which user is associated with the captured image. In an example, the image processor 124 performs the pattern matching incorporating three steps: thinning, registration, and matching.

The image processor 124 may calculate a vein feature image after noise reduction using a thinning algorithm, during which the vein patterns may be refined to single-pixel lines. An example of thinning is shown in elements (A) and (B) of FIG. 14, e.g., as compared to the multiple-pixel-width lines in element (D) of FIG. 12 as well as in elements (C) and (D) of FIG. 14.

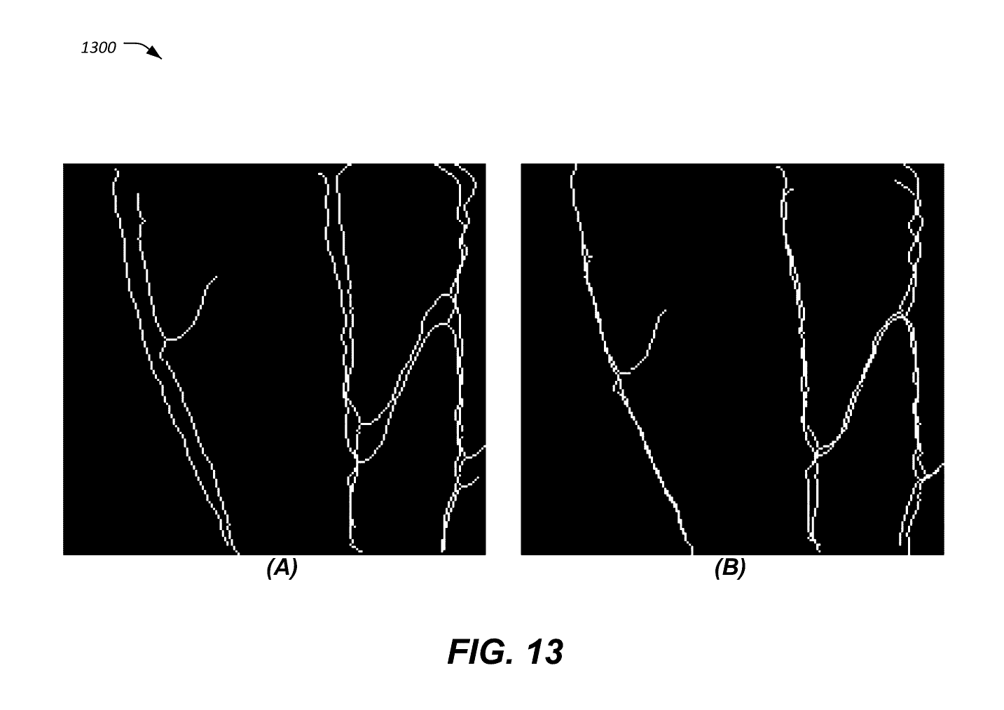

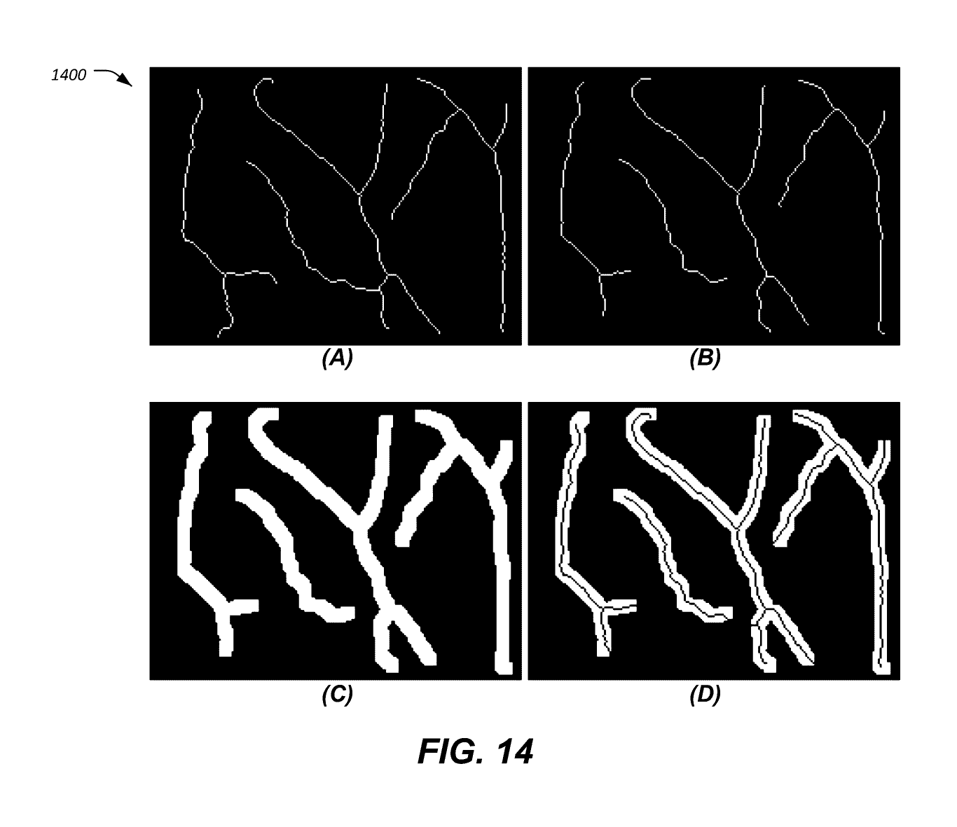

Despite the ROI segmentation procedure described above with respect to operation 404, images may still include some measure of offset brought in by slight rotation and shift. Therefore, before matching, the image processor 124 may perform a registration procedure to align vein patterns being compared. In an example, the image processor 124 may use an iterative closest bifurcation points (ICBP) algorithm to register two vein patterns, as demonstrated in the diagram 1300 of FIG. 13. FIG. 13 illustrates two thinned images to be matched.

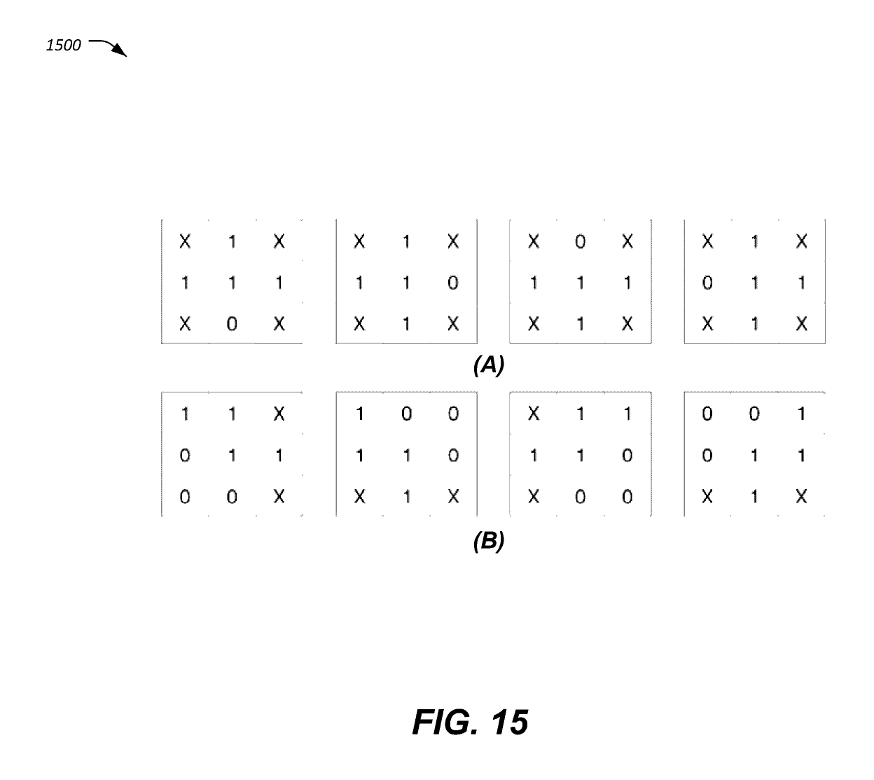

The ICBP algorithm detects bifurcation points of vessels as the input of the iterative closest point (ICP) algorithm (ICBP), which greatly increase the speed of the algorithm and improve the accuracy. For instance, the image processor 124 may utilize an 8-connected neighborhoods judgment for extracting crosspoints. After thinning the image, let the value of pixels in the background be referred to as 0 and the value of pixels of vessels be referred to as 1. Before the extraction process, patterns such as shown in FIG. 15 may be used to remove the non-single pixel point, e.g., that the pixel in the center should be removed.

For any point p1, the number of pixel representing vessels in the 8-connected neighborhoods may be defined as:

$${S}_{n} ( p 1 ) = {\sum }_{i = 2}^{9} {p}_{i} \qquad ( 2 )$$

and the number of cross points inside the 8-connected neighborhood may be defined as:

$${c}_{n} ( p 1 ) = \frac{1}{2} {\sum }_{i = 2}^{9} {p}_{i} \qquad ( 3 )$$

A point may be determined to be a bifurcation point if:

p1=1,cₙ(p1)=3 and sₙ(p1)=3 (1)

p1=1,cₙ(p1)=4 and sₙ(p1)=4 (2)

P={p₁, p₂ . . . pₙ} and Q={q₁, q₂ . . . qₙ} denote the closest pairs of extracted bifurcation points from the two different point sets, which may be saved as corresponding points. Denote H as:

$$H = {\sum }_{i = 1}^{n} {( {q}_{i} - q )}^{′ T} {( {p}_{i} - p )}^{′} \qquad ( 4 )$$

Denote U and V as:

[U,V]=svd(H) (5)

Moreover, rotation matrix R and translation matrix T may be obtained by:

R=VU^{T} (6)

T=q−Rp (7)

P_{source} and P_{target} may denote the closest pairs of two different point sets. The above procedures may be repeated until E is minimized:

$$\begin{matrix}{P}_{source} = R ⋆ {P}_{source} + T & ( 8 ) \\ E = {\sum }_{i = 1}^{n} ( {P}_{target ( i )} = {P}_{source ( i )} ) & ( 9 )\end{matrix}$$

Element (A) of FIG. 13 shows the two thinned images from the same individual before registration, and element (B) shows the two thinned images after registration utilizing the example ICBP algorithm.

The image processor 124 may utilize a matching score to determine an objective measure of the similarity degree between two vein patterns. The matching score may denote a ratio of overlap between a thinned image and a dilated image. FIG. 14 illustrates a diagram 1400 of an example of the matching process. Elements (A) and (B) of FIG. 14 illustrate two example thinned images to be matched. Element (C) of FIG. 14 illustrates a dilated image from element (B) of FIG. 14. The matching score for the image may be defined as the overlapping ratio between element (A) of FIG. 10 and element (C) of FIG. 14, as shown in FIG. 14 as element (D).

In an example, the matching score may be calculated based on Equations 10 and 11.

$$\begin{matrix}{Score}_{i} = \frac{\sum x \sum yT ( x , y ) \& D ( x , y )}{\sum x \sum yT ( x , y )} & ( 10 ) \\ Score = ( {Score}_{1} + {Score}_{2} ) / 2 & ( 11 )\end{matrix}$$

Using the equation (1), let T₁ and T₂ represent two thinned images for matching, where their corresponding dilated images are denoted as D₁ and D₂. Matching score Score₁ and Score₂ may be calculated separately. The matching score of the two vein patterns may be obtained by averaging Score₁ and Score₂.

More specifically, the matching method may utilize the thinned image overlapping dilated thinned image, and ratio of overlapping area to total area is defined as matching score. Denote I₁ as the image just taken, and T₁ as the thinned I₁. Denote I₂ and T₂ denote the as one of the original templates and its corresponding thinned image in the database respectively. Denote D₁ and D₂ as the dilated image. The procedure of the matching algorithm may then be performed as follows: Thin I₁. Register T₁ and T₂. Dilate T₁ and compute the production of five templates to obtain D₁. Dilate T₂ to obtain D₂. Use T₁, D₂ and T₂, D1 as the input of Equation (10) to obtain the matching score respectively, and Equation (11) for the final matching score between the two images.

In some examples, considering the situation in which feature points are missing due to a low quality image, to further improve accuracy the image processor 124 may step over registration and do matching again to give a final decision, only if the matching score given at the first time is lower than a pre-defined threshold.

At 410, the image processor 124 makes a decision based on the pattern matching. In an example, the decision may be an identification or verification of the image as being that of a known user. If the user is identified, for example, the system 100 may retrieve immunization logs 128 or other records relating to the identified individual. After operation 410, the process 400 ends.

Thus, hand dorsal images may be used to determine vein patterns for recognition of users. In an example, the system 100 may be used for immunization record-keeping during Polio supplemental immunization activities (SIAs) and Rubella immunization (RI). As patients are first added to the system, a hand dorsal may be taken, and stored in the database 120 as a reference information 126 image. When a repeat patient returns, a new hand dorsal image may be taken, and compared against the reference information 126 to identify the patient. By identifying the patient record at a return visit, immunization logs 128 of the patients may be retrieved and also updated for the patient. Moreover, the process may be performed without requiring the users to memorize a password or provide an identification card or other token.

Computing devices described herein generally include computer-executable instructions, where the instructions may be executable by one or more processors. Computer-executable instructions may be compiled or interpreted from computer programs created using a variety of programming languages and/or technologies, including, without limitation, and either alone or in combination, Java™, C, C++, C#, Visual Basic, Java Script, Perl, Python, PHP, Matlab, etc. In general, a processor (e.g., a microprocessor) receives instructions, e.g., from a memory, a computer-readable medium, etc., and executes these instructions, thereby performing one or more processes, including one or more of the processes described herein (e.g., the processes illustrated in FIGS. 4-5, etc.). Such instructions and other data may be stored and transmitted using a variety of computer-readable media.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system comprising:
an infrared camera;
an infrared light source; and
a processor, programmed to
receive, from the infrared camera, an image of a hand illuminated using the infrared light source, and
send the image to a remote server to identify a user corresponding to the hand according to a vein pattern of the hand.

2. The system of claim 1, wherein the infrared camera includes an infrared filter for eliminating interference from light sources other than the infrared light source.

3. The system of claim 2, wherein the infrared filter is located between a lens and a complementary metal-oxide-semiconductor (CMOS) sensor of the infrared camera.

4. The system of claim 2, wherein the infrared filter is an 850 nanometer infrared cut filter.

5. The system of claim 1, wherein the processor is further programmed to access immunization records for the user.

6. The system of claim 1, wherein the infrared light source is an infrared flashlight having a plurality of illumination intensity settings.

7. The system of claim 1, wherein the infrared camera and processor are integrated components of a mobile device.

8. The system of claim 7, wherein the infrared light source is an integrated component of the mobile device.

9. A method comprising:
receiving, from an infrared camera, an image of a hand illuminated using an infrared light source;
performing region-of-interest segmentation on the image to generate a segmented image of consistent hand location and orientation;
performing feature extraction of the segmented image to generate a feature-extracted vein image; and
matching the feature-extracted vein image against a database of feature-extracted vein images to identify a user corresponding to the hand.

10. The method of claim 9, further comprising receiving the image, over a communication network, from a transceiver of a mobile device including the infrared camera.

11. The method of claim 9, further comprising:
converting the segmented image into a grayscale image;
employing a Multi-scale Gaussian Matched Filter to extract vein pattern lines from the segmented image; and
performing binarization on the vein pattern lines to generate a binary image.

12. The method of claim 11, further comprising employing a de-noise algorithm for noise reduction of the binary image.

13. The method of claim 9, further comprising accessing a database to retrieve immunization records for the user corresponding to the hand.

14. The method of claim 9, further comprising applying an infrared filter to the infrared camera to eliminate interference from light sources other than the infrared light source.

15. The method of claim 9, further comprising calculating the feature-extracted using a thinning algorithm refining vein patterns to single-pixel lines.

16. A system comprising:
a mobile device, including a processor and a memory, the mobile device programmed to execute instructions stored to the memory to:
receive, from an infrared camera, an image of a hand illuminated using an infrared light source;
perform region-of-interest segmentation on the image to generate a segmented image of consistent hand location and orientation;
perform feature extraction of the segmented image to generate a feature-extracted vein image; and
match the feature-extracted vein image against a database of feature-extracted vein images to identify a user corresponding to the hand.

17. The system of claim 16, wherein the infrared camera includes an infrared filter for eliminating interference from light sources other than the infrared light source, the infrared filter being an 850 nanometer infrared cut filter located between a lens and a complementary metal-oxide-semiconductor (CMOS) sensor of the infrared camera.

18. The system of claim 16, wherein the infrared light source is an infrared flashlight having a plurality of illumination intensity settings.

19. The system of claim 16, wherein the infrared camera is an integrated component of the mobile device.

20. The system of claim 16, wherein the infrared light source is an integrated component of the mobile device.
